(19) United States
(12) Patent Application Publication
(10) Pub. No.: US 2026/0041562 A1
(43) Pub. Date: Feb. 12, 2026

(54) SPINAL IMPLANT DEVICE

(71) Applicant: Spineology Inc., St. Paul, MN (US)

(72) Inventors: Kyle Wolff, St. Paul Park, MN (US); John Liebelt, Lonsdale, MN (US); Aaron Weinard, Minnetonka, MN (US); Ian Malinowski, Henderson, MN (US)

(21) Appl. No.: 19/309,285

(22) Filed: Aug. 25, 2025

Related U.S. Application Data
(63) Continuation-in-part of application No. PCT/US2024/036184, filed on Jun. 28, 2024.
(60) Provisional application No. 63/523,902, filed on Jun. 28, 2023.

(51) Int. Cl. A61F 2/44; A61F 2/30

(52) U.S. Cl. CPC A61F 2/442; A61F 2002/30019; A61F 2002/444; A61F 2002/4495

(57) ABSTRACT

A spinal implant is provided that is configured to receive a fill material is provided that includes a wall that defines an interior chamber. The wall has an interior side, an exterior side, and a wall thickness that extends between the interior side and the exterior side. The wall has a porosity that permits fluid passage through the wall thickness and permits blood vessels and fibrous tissue to extend through the wall thickness. The interior chamber has a first chamber segment and a second chamber segment. In the first chamber segment, the wall has a first wall configuration. In the second chamber segment, the wall has a second wall configuration. The first chamber segment has a first volumetric expansion ratio and the second chamber segment has a second volumetric expansion ratio. The second volumetric expansion ratio is greater than the first volumetric expansion ratio.

### BACKGROUND OF THE INVENTION

### 1. Technical Field

The present invention generally relates to implantable devices used in orthopedic surgeries, and more particularly to devices and methods used in spinal interbody fusion procedures.

### 2. Background Information

A spinal disc consists of three parts: a nucleus pulposis (the “nucleus”) which is a central portion that functions as a compression-resisting cushion, an annulus fibrosis (the “annulus”) which is a peripheral portion that functions as a tension-resisting hoop, and third the cartilaginous end plates (the “end plates”) that form the superior and inferior borders of the disc, consisting of the upper and lower surfaces of the vertebral body bones adjacent to the disc. Many studies have concluded that mechanical back pain is the most common and costly musculoskeletal condition affecting aging humans in modern societies. Mechanical back pain may be caused by several factors. Evidence suggests that degeneration of the spinal intervertebral disc is the most common condition causing stenosis, radiculopathy, and instability, any of which may lead to symptoms such as back and radiating limb pain.

Many devices have been invented for the purpose of easing the pain associated with degenerative disc disease. There is, however, a continuing need to provide improved devices adapted for treatment of degenerative disc disease.

### SUMMARY

A spinal implant that is configured to receive a fill material is provided that includes a wall that defines an interior chamber. The wall has an interior side, an exterior side, and a wall thickness that extends between the interior side and the exterior side. The wall has a porosity that permits fluid passage through the wall thickness and permits blood vessels and fibrous tissue to extend through the wall thickness. The interior chamber has a first chamber segment and a second chamber segment. In the first chamber segment, the wall has a first wall configuration. In the second chamber segment, the wall has a second wall configuration. The first chamber segment has a first volumetric expansion ratio, and the second chamber segment has a second volumetric expansion ratio. The second volumetric expansion ratio is greater than the first volumetric expansion ratio.

In any of the aspects or embodiments described above and herein, the first volumetric expansion ratio may be a first chamber segment fill expanded state volume over a first chamber segment unfilled state volume, and the second volumetric expansion ratio may be a second chamber segment fill expanded state volume over a second chamber segment unfilled state volume.

In any of the aspects or embodiments described above and herein, the first chamber segment fill expanded state volume may be a volume of the first chamber segment when the first chamber segment is subject to a first pressure produced by a fill material disposed within the first chamber segment. The second chamber segment fill expanded state volume may be a volume of the second chamber segment when the second chamber segment is subject to a second pressure produced by the fill material disposed within the second chamber segment. The first pressure may equal the second pressure.

In any of the aspects or embodiments described above and herein, the implant may be disposable in a collapsed state and in a fill expanded state.

In any of the aspects or embodiments described above and herein, the first wall configuration may be a first mesh of strands, and the second wall configuration may be a second mesh of strands, and the first mesh of strands may be differently configured than the second mesh of strands.

In any of the aspects or embodiments described above and herein, the first mesh of strands may have a first Jersey knitting stitch configuration, and the second mesh of strands may have a second Jersey knitting stitch configuration.

In any of the aspects or embodiments described above and herein, the first Jersey knitting stitch configuration may have a first set of stitch parameters and the second Jersey knitting stitch configuration may have a second set of stitch parameters, and the first set of stitch parameters may be different than the second set of stitch parameters.

In any of the aspects or embodiments described above and herein, the first mesh of strands may be a first knitting stitch configuration, and the second mesh of strands may be a second knitting stitch configuration.

In any of the aspects or embodiments described above and herein, the first mesh of strands may comprise a first strand material, and the second mesh of strands may comprise a second strand material different than the first strand material.

In any of the aspects or embodiments described above and herein, the first chamber segment may be a posterior chamber segment, and the second chamber segment may be an anterior chamber segment.

In any of the aspects or embodiments described above and herein, the first chamber segment may be a first lateral chamber segment, and the second chamber segment may be a second lateral chamber segment, and the second lateral chamber segment may be disposed opposite the first lateral chamber segment.

According to an aspect of the present disclosure, a spinal implant that is configured to receive a fill material is provided. The spinal implant includes a wall that defines an interior chamber. The wall has an interior side, an exterior side, and a wall thickness that extends between the interior side and the exterior side. The wall has a porosity that permits fluid passage through the wall thickness and permits blood vessels and fibrous tissue to extend through the wall thickness. The interior chamber has a first chamber segment and a second chamber segment. In the first chamber segment, the wall has a first mesh of strands configuration, and in the second chamber segment the wall has a second mesh of strands configuration.

In any of the aspects or embodiments described above and herein, the first mesh of strands configuration may be a first knitting stitch configuration, and the second mesh of strands configuration may be a second knitting stitch configuration that is different than the first knitting stitch configuration.

In any of the aspects or embodiments described above and herein, the first chamber segment may have a first volumetric expansion ratio that is a function of the first mesh of strands configuration, and the second chamber segment may have a second volumetric expansion ratio that is a function of the second mesh of strands configuration, and the first volumetric expansion ratio may be different than the first volumetric expansion ratio.

According to an aspect of the present disclosure, a spinal implant system is provided that includes a fill material and an implant. The implant comprises a wall that defines an interior chamber. The wall has an interior side, an exterior side, and a wall thickness that extends between the interior side and the exterior side. The wall has a porosity that permits fluid passage through the wall thickness and permits blood vessels and fibrous tissue to extend through the wall thickness. The interior chamber has a first chamber segment and a second chamber segment. In the first chamber segment, the wall has a first wall configuration. In the second chamber segment, the wall has a second wall configuration. The first chamber segment is configured to expand by a first volumetric expansion ratio when the fill material is packed into the first chamber segment in a manner that applies a first pressure to the wall within the first wall chamber, and the second chamber segment is configured to expand by a second volumetric expansion ratio when the fill material is packed into the second chamber segment in a manner that applies a second pressure to the wall within the second wall chamber, wherein the first pressure substantially equals the second pressure.

The foregoing features and elements may be combined in various combinations without exclusivity, unless expressly indicated otherwise. For example, aspects and/or embodiments of the present disclosure may include any one or more of the individual features or elements disclosed above and/or below alone or in any combination thereof. These features and elements as well as the operation thereof will become more apparent in light of the following description and the accompanying drawings. The following description and drawings are intended to be exemplary in nature and non-limiting.

### BRIEF DESCRIPTION OF THE DRAWINGS

FIG. 1 is a diagrammatic representation of a superior vertebra, an inferior vertebra, and a disc disposed therebetween.

FIG. 2 is a diagrammatic perspective view of a present disclosure spinal implant embodiment.

FIG. 3 is a diagrammatic side view of a present disclosure spinal implant embodiment.

FIG. 4 is a diagrammatic sectional view of the spinal implant embodiment shown in FIG. 3 along sectional lines 4-4.

FIG. 5 is a diagrammatic representation of a superior vertebra, an inferior vertebra, and a disc disposed therebetween with a present disclosure multi-chamber spinal implant embodiment disposed in a cavity in the disc, with the implant shown in a collapsed state.

FIG. 6 is a diagrammatic representation of a superior vertebra, an inferior vertebra, and a disc as shown in FIG. 6 with the present disclosure multi-chamber spinal implant embodiment disposed in the disc cavity in an expanded state.

FIG. 7 is a diagrammatic representation of a Jersey knitting stitch.

### DETAILED DESCRIPTION

The present disclosure is directed to a spinal implant device and system that may be utilized in an interbody fusion procedure and a method for using the same. As will be disclosed in greater detail herein, the spinal implant is configurable in a collapsed state and in a fill expanded state. The ability of the spinal implant to be in a collapsed state facilitates its use within an interbody fusion procedure, including use within a percutaneous interbody fusion procedure. The present disclosure is not limited to use within any particular interbody fusion procedure. The spinal implant may be transitioned from a collapsed state to a fill expanded state by inserting a fill material into an interior chamber of the implant. The present disclosure spinal implant is understood to provide a clinician with substantially greater ability to produce a desired orientation between adjacent spinal vertebrae and the benefits attendant therewith.

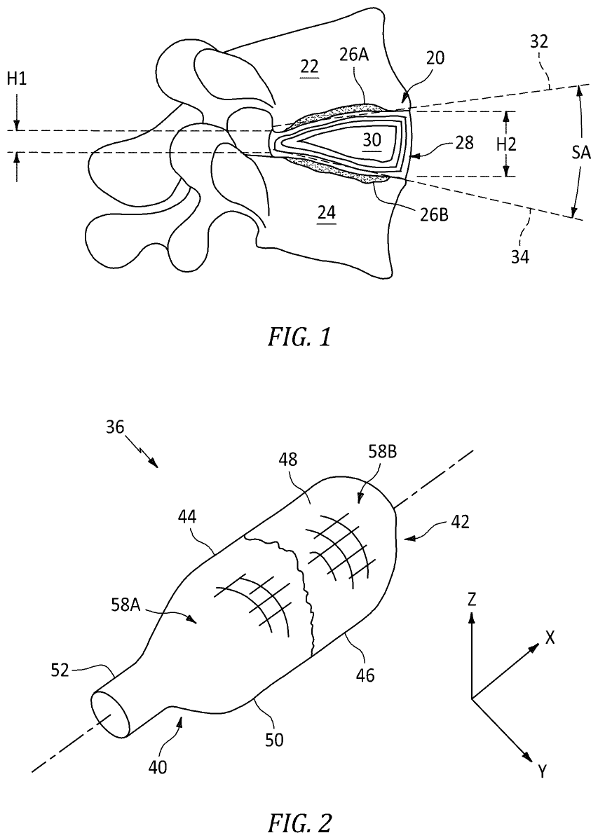

FIG. 1 diagrammatically illustrates a disc 20 disposed between a superior vertebra 22 and an inferior vertebra 24. The disc 20 is sectionally illustrated to show the inferior endplate 26A of the superior vertebra 22, the superior endplate 26B of the inferior vertebra 24, the annulus 28, and the nucleus 30. FIG. 1 includes a first plane 32 that is representative of the inferior endplate 26A of the superior vertebra 22 and a second plane 34 that is representative of the superior endplate 26B of the inferior vertebra 24. The included angle between the first and second planes 32, 34 is referred to hereinafter as the segmentation angle (“SA”). It should be noted that the lordosis angle of a spinal region (e.g., the lumbar region or the cervical region) is understood to be a measure of a collective curvature of a respective spinal region. It is further understood that there are other measurements of lordosis angle aspects (e.g., segmental lordosis, cumulative lordosis, and the like) that are sometimes used to refer to aspects of a lordosis angle. For sake of clarity, the term “segmentation angle” (which may contribute to the lordosis angle) is used herein to refer to the angle “SA” disposed within the sagittal plane defined by a first and second planes 32, 34. FIG. 1 also includes a disc height value “H1” disposed adjacent the posterior end of the disc 20 and a disc height value “H2” disposed adjacent the anterior end of the disc 20. The difference in disc height (H2>H1) and the segmentation angle “SA” illustrates the normal asymmetry of the disc 20, giving the disc 20 a “trapezoid-like” cross-sectional geometry.

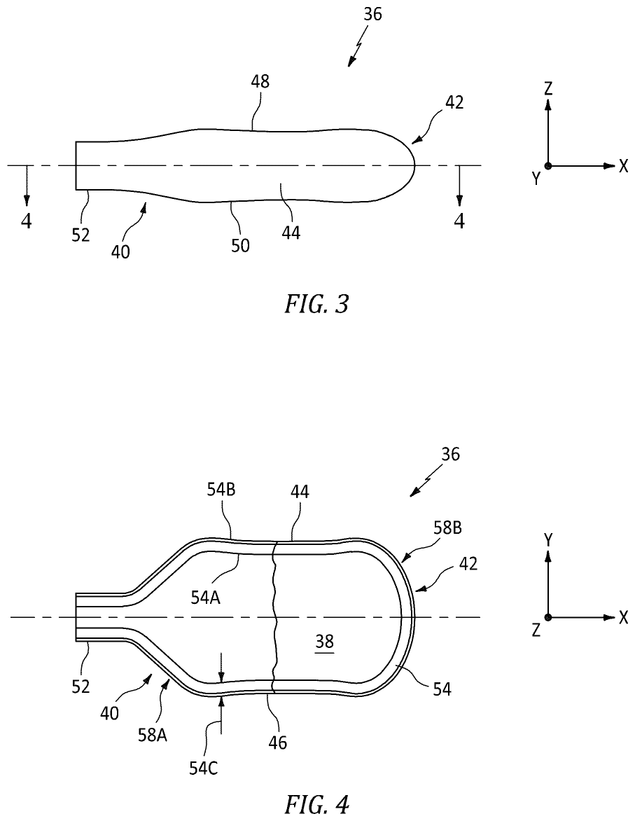

FIG. 2 is a diagrammatic perspective illustration of an unfilled present disclosure spinal implant 36 embodiment. FIG. 3 is a diagrammatic side view of the implant 36 embodiment like that shown in FIG. 2. FIG. 4 is a diagrammatic sectional view of the implant 36 embodiment shown in FIG. 3 along the sectional line 4-4.

Referring to FIG. 4, the spinal implant 36 is a unitary structure that includes an interior chamber 38 and may be described as having a posterior end 40, an anterior end 42, a first lateral side 44, a second lateral side 46, a superior side 48, and an inferior side 50. In some embodiments (e.g., like that shown in FIG. 2), the spinal implant 36 may include a fill port 52 that provides a passage into the interior chamber 38 of the implant 36. The present disclosure implants 36 do not require a fill port 52, however. To facilitate the description herein, the spinal implant 36 will be described herein as having a length (e.g., X-axis) that extends between the posterior end 40 and the anterior end 42, a width (e.g., Y-axis) that extends between the first lateral side 44 and the second lateral side 46, and a height (e.g., Z-axis) that extends between the superior side 48 and the inferior side 50.

The fill port 52 (if included) may be disposed in an open configuration and in a closed configuration. In the open configuration, the fill port 52 allows an instrument (e.g., a tube—not shown) to be inserted into the interior chamber 38 of the implant 36. In the closed configuration, the fill port 52 does not allow the escape of fill material from the interior chamber 38 via the fill port 52. In some instances, the spinal implant 36 may default to a closed configuration when the tube is withdrawn from the fill port 52; i.e., no further action is required to achieve the closed configuration. In other embodiments, the fill port 52 may be placed in a closed configuration by a suturing process, or by an adhesive, or by welding, or the like. The present disclosure is not limited to any particular fill port 52 configuration or fill port 52 closing procedure. As stated above, in some embodiments a fill port 52 may not be included. For example, the implant wall may be configured to allow a fill device (not shown; e.g., a catheter, a needle, a fill tube, or the like) to insert fill material through the wall 54 and into the interior chamber 38. Once the interior chamber 38 is filled and the fill device is removed, the wall 54 returns to its original form that prevents fill material to pass therethrough.

Referring to FIG. 4, the interior chamber 38 of the implant 36 is defined by a continuous wall 54 or by a plurality of walls 54 that collectively define the interior chamber 38. In those embodiments wherein the interior chamber 38 is defined by a single continuous wall 54, the interior chamber 38 may be described as having wall segments that are portions of the single continuous wall 54. The wall 54 includes an interior side 54A, an exterior side 54B, and a thickness 54C that extends between the opposing interior and exterior surfaces 54A, 54B.

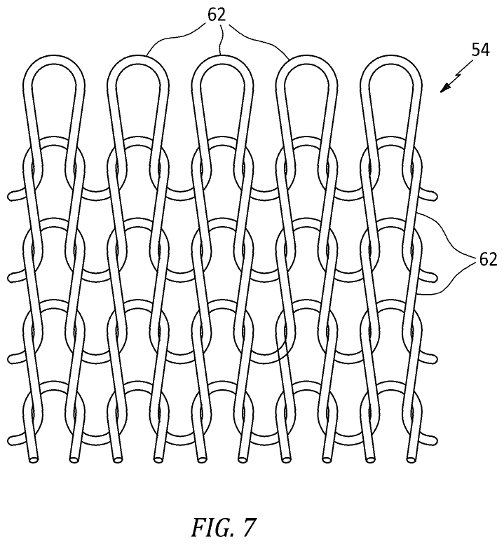

The wall 54 of the present disclosure spinal implant 36 is both porous and pliable. The porosity of the wall 54 is chosen to permit the passage of fluids and solutions through the wall 54 while also preventing fill material 56 (see FIG. 6) from passing through the wall 54. The passage of fluids and solutions into and out of the interior chamber 38 is understood to facilitate the ingrowth, on-growth, and through-growth of blood vessels and fibrous tissue and bony trabeculae and consequently promote fusion between a fill material 56 (See FIG. 6) disposed within the interior chamber 38 and the vertebral endplates 26A, 26B. The size (i.e., cross-sectional area) of the pores that create the porous nature of the wall 54 may be chosen based on the fill material 56 used within the spinal implant 36; e.g., based on the particulate size(s) within the fill material 56. The size of the pores within a wall 54 of the implant 36 may be uniform throughout the entirety of the respective wall 54, but that is not required. In some embodiments, the entirety of the implant wall 54 is porous. In some embodiments, less than all of the wall 54 is porous.

Referring to FIG. 7, in some embodiments, the wall 54 of the present disclosure spinal implant 36 may be configured as a mesh of strands 62; i.e., an interlaced arrangement of strands 62. As will be detailed herein, a non-limiting example of an interlaced arrangement of strands is a knitting stitch; e.g., a Jersey knitting stitch as diagrammatically shown in FIG. 7.

The strands may comprise biocompatible materials. For example, a strand material used for medical sutures may be used to form the mesh. Other non-limiting examples of acceptable mesh strand materials include titanium and other biocompatible metals in various material configurations, nitinol, biodegradable materials, polymeric materials, and others. In some embodiments, mesh strands may be treated (e.g., by coating, absorbance, or the like) with a bioactive solution such as one containing an antibiotic, a pharmaceutical agent, osteoconductive material, or a bone morphogenic protein such as, for example, recombinant human bone morphogenetic protein (rhBMP), and any combinations thereof. U.S. Patent Publication No. 2008/0113008, entitled “Absorbent Fabric Implant”, which is hereby incorporated by reference in its entirety, discloses examples of bioactive materials that may be utilized with the present disclosure spinal implant 36. The present disclosure is not limited to any particular mesh strand materials, other than one that is suitable for the application at hand.

As indicated herein, the spinal implant 36 may be disposed in a collapsed state, an unfilled state, or a fill expanded state. In a collapsed state, the volume of the implant interior chamber 38 may be zero (or nearly zero); e.g., the interior sides 54A of the wall 54 (or wall segments) that define the interior chamber 38 are substantially in contact with one another thereby producing a zero volume interior chamber 38. In an unfilled state, the interior sides 54A of the wall 54 are spaced apart from one another and collectively define an “at rest” interior chamber 38 of the spinal implant 36. In the unfilled state, the wall 54 of the spinal implant 36 is at rest; i.e., the wall 54 is not under tension and no force is acting on the wall 54 that would cause the wall 54 to elongate or expand (i.e., deviate from its at rest configuration) and consequently cause the interior chamber 38 to volumetrically increase in size. The term “fill expanded state” as used herein refers to an implant 36 configuration wherein the interior chamber 38 is at a volume resulting from a pressure being applied to the interior side 54A of the wall 54. For example, and as detailed herein, if sufficient fill material 56 is packed into the interior chamber 38, the fill material 56 will produce a pressure force acting on the interior side 54A of the implant wall 54. That pressure force may (as will be described herein) cause portions of the interior chamber 38 to increase volumetrically.

The interior chamber 38 of the present disclosure implant 36 includes at least a first chamber segment 58A and a second chamber segment 58B, with each chamber segment 58A, 58B having a different wall configuration. The term “wall configuration” as used herein refers to the physical characteristics of the wall 54 itself and does not refer to the geometry in which the wall 54 is disposed. Hence, a first wall and a second wall may have the same geometry and still have different wall configurations. As will be described in detail herein, as a result of the respective wall configurations, one of the chamber segments 58A, 58B is configured to volumetrically expand a greater percentage than the other chamber segment 58B, 58A when the walls 54 of each chamber segment 58A, 58B are subjected to the same pressure force; a pressure force acting on the walls from the interior chamber 38. In this manner, the size of one chamber segment 58A, 58B may be increased disproportionately relative to another chamber segment 58B, 58A during the filling process, which may facilitate establishing a desired separation between inferior and superior vertebra 22, 24 and a desired segmentation angle therebetween.

Referring to FIGS. 2-4, to facilitate the description herein, the first chamber segment 58A may be referred to as a posterior chamber segment that is disposed adjacent the posterior end 40 of the spinal implant 36 and the second chamber segment 58B may be referred to as an anterior chamber segment disposed adjacent the anterior end 42 of the spinal implant 36. To be clear, the present disclosure implant 36 is not limited to an embodiment having only a posterior chamber segment and an anterior chamber segment. The chamber segments 58A, 58B (e.g., the posterior and anterior chamber segments) may be open to one another; i.e., no barrier is disposed between the posterior and anterior chamber segments that would inhibit the passage of fill material therebetween. The posterior chamber segment may be described as having a first wall configuration and the anterior chamber segment may be described as having a second wall configuration, wherein the second wall configuration is different than the first wall configuration. In some embodiments, the plurality of chamber segments 58A, 58B may each have the same unfilled state volume; e.g., both the posterior chamber segment and the anterior chamber segment have an unfilled state volume equal to 50% of the total interior chamber unfilled state volume. In some embodiments, the chamber segments may have unequal unfilled state volumes; e.g., the posterior chamber segment may have an unfilled state volume equal to 33% of the total interior chamber unfilled state volume, and the anterior chamber segment may have an unfilled state volume equal to 67% of the total interior chamber unfilled state volume.

In those embodiments that include a wall 54 configured as a mesh of strands (e.g., a type of wall configuration), the wall 54 may include mesh arrangements that allow one chamber segment 58A, 58B to volumetrically expand a greater percentage than another chamber segment 58B, 58A when the interior side of each chamber segment is subjected to the same pressure force. The “same pressure force” assumes that the fill material 56 packed into the chamber segments 58A, 58B is substantially uniformly packed throughout the interior chamber 38 thereby resulting in substantially the same pressure force being applied to the wall 54 of the spinal implant 36 throughout the chamber segments 58A, 58B. Hence, differences in volumetric expansion are not a result of non-uniform fill material packing. A “volumetric expansion” for a given chamber segment 58A, 58B may occur via the mesh expanding, or the mesh strands elongating, or the like, or any combination thereof, as a result of the pressure force applied by the fill material 56.

The volumetric expansion of an implant chamber segment 58A, 58B between an unfilled state and a fill expanded state may be described in terms of a volumetric expansion ratio. The volumetric expansion ratio (VER) may be defined as follows:

$$VER = \frac{Chamber Segment Volume in Fill Expanded State}{Chamber Segment Volume in Unfilled State}$$

If a chamber segment 58A, 58B does not expand at all from the chamber segment volume in an unfilled state to the chamber segment volume in a fill expanded state, the VER would have a value of one (1.0). A VER value of 1.1 would mean that the chamber segment volume in a fill expanded state is 10% greater than the chamber segment volume in an unfilled state, a VER value of 1.25 would mean that the chamber segment volume in a fill expanded state is 25% greater than the chamber segment volume in an unfilled state, and so on.

Different mesh strand patterns (i.e., different wall configurations) may have different expansion characteristics (expansion characteristics are sometimes referred to as “extension” characteristics). As an example, a Jersey knitting stitch may extend (e.g., stretch) more when under tension than an interlocking knitting stitch made from the same strand material. Hence, different stitch patterns (i.e., different wall configurations) may result in different volumetric expansion characteristics. Volumetric expansion for a given chamber segment 58A, 58B may also occur by other wall configuration means; e.g., mesh elongation, inclusion of different material strands, mesh directional orientation, and the like.

There are several ways volumetric expansion ratio (VER) differences between chamber segments 58A, 58B can be accomplished in a spinal implant 36. For example, a first VER may be produced by using a first mesh arrangement (i.e., a first wall configuration) within a first chamber segment 58A and using a second mesh arrangement (i.e., a second wall configuration) within a second chamber segment 58B. The first mesh arrangement may be achieved using a first Jersey stitch configuration and the second mesh arrangement may be achieved using a second Jersey stitch configuration that is different from the first Jersey stitch configuration. The specific differences between the first and second Jersey stitch configurations may lie in stitch parameters. For example, the tallness (or the “height”) of the stitch courses along a length of the mesh arrangement may be varied between the first and second Jersey stitch configurations. As another example, the needles per course may be varied between the first and second Jersey stitch configurations. In this example, the second mesh arrangement of the second chamber segment 58B may be configured for greater volumetric expansion than the first mesh arrangement of the first chamber segment 58A. The present disclosure is not limited to these example stitch parameter variations within a given stitch type. Another example of creating VER differences between chamber segments 58A, 58B, includes using a first stitch type in a first chamber segment and a second stitch type in a second chamber segment, wherein the first stitch type is different than the second stitch type. For example, a first stitch type (e.g., an interlock stitch) may be used in a first chamber segment 58A and a second stitch type (e.g., a Jersey stitch) may be used in a second chamber segment 58B. In this example, the expansion characteristic of the interlock stitch may be less than the expansion characteristic of the Jersey stitch. Hence, for a given uniform amount of pressure applied to the first chamber segment 58A (comprising the interlock stitch), the first chamber segment 58A will expand less than the second chamber segment 58B (comprising the Jersey stitch). The present disclosure is not limited to any particular means for creating volumetric expansion ratio (VER) differences between chamber segments in an implant 36.

The fill material 56 used to fill the implant 36 may include one or more of the following, or any other biocompatible material judged to have the desired physiologic response, or any combination thereof: demineralized bone material, morselized bone graft, cortical, cancellous, or cortico-cancellous, including autograft, allograft, or xenograft; any bone graft substitute or combination of bone graft substitutes, or combinations of bone graft and bone graft substitutes, or bone inducing substances, including but not limited to: calcium phosphates, calcium sulfates, calcium carbonates, hydroxyapatite, bone morphogenic proteins, calcified and/or decalcified bone derivatives; bone cements, such as injectable ceramic and polymethylmethacrylate bone cements, or any osteoconductive biocompatible material known to promote bone formation, titanium and other biocompatible metals in various material configurations, resorbable metals such as magnesium (Mg) and zinc (Zn), and polymeric particles including but not limited to polyether ether ketone (PEEK), polyether ketone ketone (PEKK), polyethylene (PE), polyurethane (PU), polycarbonate polyurethane (PCU), polylactic acid (PLA), polyamide (PA), poly lactic-co-glycolic acid (PLGA), and polyglycolide acid (PGA). The present disclosure is not limited to any particular type of fill material 56.

The present disclosure spinal implant 36 is understood to provide an improved capability to produce a desired orientation between a superior vertebra 22 and an inferior vertebra 24; e.g., vertebrae separation distance and/or segmentation angle. Specifically, the variable expansion between implant chamber segments 58A, 58B is understood to give the surgeon performing the procedure a greater ability to customize the spinal implant 36 to create the desired vertebral orientation; e.g., vertebrae separation distance and segmentation angle. In addition, the configuration of the present disclosure spinal implants 36 (i.e., customizable chamber segments 58A, 58B) is understood to allow a surgeon to choose a spinal implant 36 having a chamber segment volumetric expansion ratio that best suits the procedure at hand. The present disclosure spinal implant is also understood to provide an improvement in conformity between the spinal implant and the respective endplate 26A, 26B. The improved conformity is understood to facilitate the amount of fusion between the endplates and the spinal implant; e.g., by increasing the amount of area in contact between the spinal implant and the respective endplate 26A, 26B.

As indicated herein, the present disclosure spinal implant 36 may be utilized in an interbody fusion procedure to alleviate issues associated with a medical condition such as degenerative disc disease. The procedure that may be used to insert the spinal implant 36 may be chosen based on numerous factors including which spinal vertebrae are to be fused, the reason for the spinal fusion, and the general health and body shape of the patient. Some traditional interbody fusion procedures (“open surgery”) require direct visualization by the clinician and may require bone to be cut and significant retraction of soft tissue and nerve roots. Alternatively, it may be possible to perform a minimally invasive form of interbody fusion often referred to as percutaneous spinal interbody fusion. The present disclosure spinal implant 36 may be used in either of these types of interbody fusion procedures (and others), and as will be detailed herein can provide significant clinical benefit to the patient. To facilitate the description herein, an example of how the present disclosure spinal implant 36 may be used in a percutaneous spinal interbody fusion is provided.

Percutaneous interbody fusion is performed under indirect visualization using x-ray or other imaging and/or navigation technologies, including robotics and endoscopy. Because neural tissue cannot be seen on x-ray, active neural monitoring may be used to avoid nerve damage that may otherwise occur during the procedure. There are two types of neural monitoring that are generally used in spine surgery: electromyography (EMG) and somatosensory evoked potential (SSEP). When using neural monitoring in the spine, the surgeon evaluates nerve potential by checking for evoked responses. An instrument, such as a neural stimulating component, may be used to mechanically manipulate or electrically stimulate the nerve in order to evoke a response.

Dilators may be used with the neural monitoring to incrementally establish an acceptable approach path and cannula for the surgical instruments used in the interbody fusion. The dilation step includes penetrating the surface of the annulus portion of the target disc. The orientation of the approach path relative to the patient's spine (e.g., an anterior approach, a posterior approach, a lateral approach, a posterolateral approach, an anterolateral approach, and so on) may be chosen by the clinician based on the circumstances of the patient. It is understood that a variety of different approaches may be used in a percutaneous interbody fusion using a present disclosure spinal implant 36. Once the acceptable approach path and cannula are established and the annulus portion of the target disc 20 is penetrated, a cavity is created in the nucleus of the disc 20 by removing at least a portion of disc nucleus. The disc removal process may include liberating disc nucleus material (e.g., by cutting and scraping) using a tool having one or more blades. The liberated disc nucleus material may be removed using a rongeur or the like. The liberation and removal of disc material creates the cavity. The geometry of the cavity may be chosen in view of the procedure at hand and the geometry of the desired implant. The disc cavity preparation process may include preparing a fusion bed of bleeding bone at the vertebral endplates to facilitate new bone growth for fusion to occur. The present disclosure is not limited to any particular disc cavity preparation process. Once the cavity and the endplate fusion beds are established, the present disclosure spinal implant 36 in a collapsed state may be inserted percutaneously.

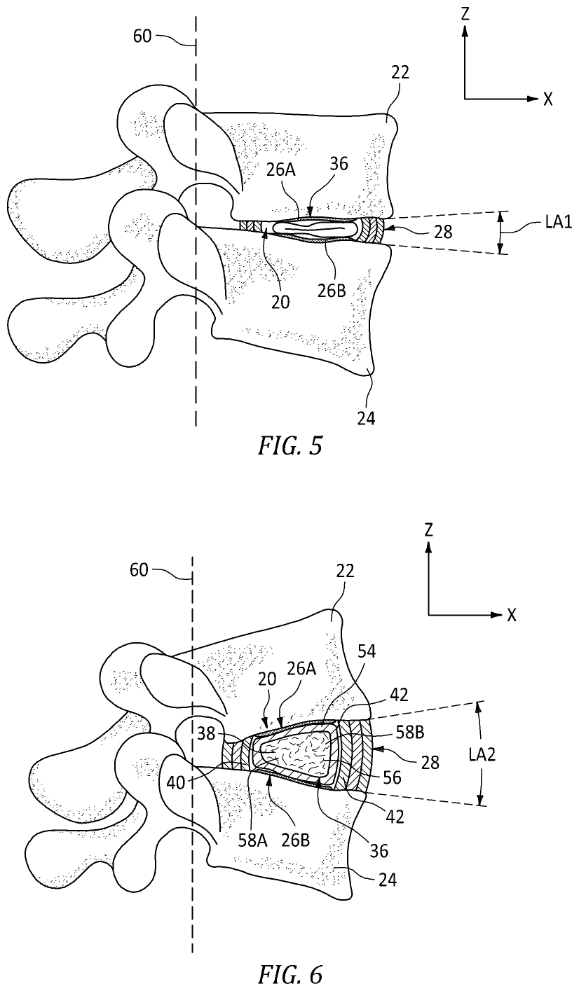

FIG. 5 diagrammatically illustrates a pair of spinal vertebrae (e.g., a superior vertebra 22 and an inferior vertebra 24) with a disc 20 disposed therebetween. In this view, the disc 20 is shown diagrammatically sectioned to facilitate the view and the description of the end plates 26A, 26B, the annulus 28, a cavity where the disc nucleus (not shown) resided prior to being excised, and a present disclosure spinal implant 36. In FIG. 5, the implant 36 is disposed in the cavity in a collapsed state and the inferior endplate 26A of the superior vertebra 22 is almost parallel to the superior endplate 26B of the inferior vertebra 24. Hence, the segmentation angle (“LA1”) between the superior vertebra 22 and the inferior vertebra 24 is minimal. The dashed vertical line 60 extending through the vertebra 22, 24 is representative of the coronal plane.

FIG. 6 diagrammatically illustrates the superior and inferior spinal vertebra 22, 24, the disc 20, and the spinal implant 36 shown in FIG. 5. In FIG. 6, the implant 36 is shown in a fill expanded state with the posterior chamber segment (i.e., first chamber segment 58A) and the anterior chamber segment (i.e., first chamber segment 58B) packed with a fill material 56. With the implant 36 in the fill expanded state, the height between the superior vertebra 22 and the inferior vertebra 24 has increased in both the posterior region and the anterior region, and the segmentation angle between the endplates 26A, 26B of the superior vertebra 22 and the inferior vertebra 24 has increased from “LA1” to “LA2” (LA2>LA1) relative to the diagrammatic view shown in FIG. 5. Here again, the dashed vertical line 60 extending through the vertebra 22, 24 is representative of the coronal plane.

In this example, the present disclosure spinal implant 36 may be configured so that one of the posterior chamber segment or the anterior chamber segment has a volumetric expansion ratio (VER) that is greater than the VER of the other of the posterior chamber segment or the anterior chamber segment. The VER of the posterior chamber segment and the VER of the anterior chamber segment of the interior chamber 38 of the spinal implant 36 may be chosen to create a desired orientation between the superior and inferior vertebra 24 in terms of vertebrae separation distance and intervertebral angle within the sagittal plane; e.g., the segmentation angle. As indicated herein, other embodiments of the present disclosure may be configured to create a desired orientation between the superior and inferior vertebra 22, 24 relative to the coronal and axial planes, or any combination of the sagittal, coronal, and axial planes.

While the principles of the disclosure have been described above in connection with specific apparatuses and methods, it is to be clearly understood that this description is made only by way of example and not as limitation on the scope of the disclosure. Specific details are given in the above description to provide a thorough understanding of the embodiments. However, it is understood that the embodiments may be practiced without these specific details.

It is noted that the embodiments may be described as a process which is depicted as a flowchart, a flow diagram, a block diagram, etc. Although any one of these structures may describe the operations as a sequential process, many of the operations can be performed in parallel or concurrently. In addition, the order of the operations may be rearranged. A process may correspond to a method, a function, a procedure, a subroutine, a subprogram, etc.

The singular forms “a,” “an,” and “the” refer to one or more than one, unless the context clearly dictates otherwise. For example, the term “comprising a specimen” includes single or plural specimens and is considered equivalent to the phrase “comprising at least one specimen.” The term “or” refers to a single element of stated alternative elements or a combination of two or more elements unless the context clearly indicates otherwise. As used herein, “comprises” means “includes.” Thus, “comprising A or B,” means “including A or B, or A and B,” without excluding additional elements.

It is noted that various connections are set forth between elements in the present description and drawings (the contents of which are included in this disclosure by way of reference). It is noted that these connections are general and, unless specified otherwise, may be direct or indirect and that this specification is not intended to be limiting in this respect. Any reference to attached, fixed, connected or the like may include permanent, removable, temporary, partial, full and/or any other possible attachment option.

No element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprise,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

The terms “substantially,” “about,” “approximately,” and other similar terms of approximation used throughout this patent application are intended to encompass variations or ranges that are reasonable and customary in the relevant field. These terms should be construed as allowing for variations that do not alter the basic essence or functionality of the invention. Such variations may include, but are not limited to, variations due to manufacturing tolerances, materials used, or inherent characteristics of the elements described in the claims and should be understood as falling within the scope of the claims unless explicitly stated otherwise. As an example, the present application describes that the term “same pressure force” assumes that the fill material packed into the chamber segments is “substantially” uniformly packed throughout the interior chamber 38 thereby resulting in substantially the same pressure force being applied to the wall 54 of the spinal implant 36 throughout the chamber segments. In this example, the term “substantially” is used to indicate that although the fill material packing may not be exactly uniform, it is packed sufficiently uniform such that the variations create inconsequential variations in the application of pressure force.

While various inventive aspects, concepts and features of the disclosures may be described and illustrated herein as embodied in combination in the exemplary embodiments, these various aspects, concepts, and features may be used in many alternative embodiments, either individually or in various combinations and sub-combinations thereof. Unless expressly excluded herein all such combinations and sub-combinations are intended to be within the scope of the present application. Still further, while various alternative embodiments as to the various aspects, concepts, and features of the disclosures—such as alternative materials, structures, configurations, methods, devices, and components, and so on—may be described herein, such descriptions are not intended to be a complete or exhaustive list of available alternative embodiments, whether presently known or later developed. Those skilled in the art may readily adopt one or more of the inventive aspects, concepts, or features into additional embodiments and uses within the scope of the present application even if such embodiments are not expressly disclosed herein. For example, in the exemplary embodiments described above within the Detailed Description portion of the present specification, elements may be described as individual units and shown as independent of one another to facilitate the description. In alternative embodiments, such elements may be configured as combined elements. It is further noted that various method or process steps for embodiments of the present disclosure are described herein. The description may present method and/or process steps as a particular sequence. However, to the extent that the method or process does not rely on the particular order of steps set forth herein, the method or process should not be limited to the particular sequence of steps described. As one of ordinary skill in the art would appreciate, other sequences of steps may be possible.

What is claimed:

1. A spinal implant configured to receive a fill material, comprising:
a wall that defines an interior chamber, wherein the wall has an interior side, an exterior side, and a wall thickness that extends between the interior side and the exterior side, wherein the wall has a porosity that permits fluid passage through the wall thickness and permits blood vessels and fibrous tissue to extend through the wall thickness; and
wherein the interior chamber has a first chamber segment and a second chamber segment, and in the first chamber segment the wall has a first wall configuration, and in the second chamber segment the wall has a second wall configuration; and
wherein the first chamber segment has a first volumetric expansion ratio and the second chamber segment has a second volumetric expansion ratio, and the second volumetric expansion ratio is greater than the first volumetric expansion ratio.

2. The spinal implant of claim 1, wherein the first volumetric expansion ratio is a first chamber segment fill expanded state volume over a first chamber segment unfilled state volume; and
wherein the second volumetric expansion ratio is a second chamber segment fill expanded state volume over a second chamber segment unfilled state volume.

3. The spinal implant of claim 2, wherein the first chamber segment fill expanded state volume is a volume of the first chamber segment when the first chamber segment is subject to a first pressure produced by a fill material disposed within the first chamber segment, and the second chamber segment fill expanded state volume is a volume of the second chamber segment when the second chamber segment is subject to a second pressure produced by the fill material disposed within the second chamber segment, wherein the first pressure equals the second pressure.

4. The spinal implant of claim 1, wherein the implant is disposable in a collapsed state and in a fill expanded state.

5. The spinal implant of claim 1, wherein the first wall configuration is a first mesh of strands, and the second wall configuration is a second mesh of strands, and the first mesh of strands is differently configured than the second mesh of strands.

6. The spinal implant of claim 5, wherein the first mesh of strands has a first Jersey knitting stitch configuration, and the second mesh of strands has a second Jersey knitting stitch configuration.

7. The spinal implant of claim 6, wherein the first Jersey knitting stitch configuration has a first set of stitch parameters and the second Jersey knitting stitch configuration has a second set of stitch parameters, and the first set of stitch parameters are different than the second set of stitch parameters.

8. The spinal implant of claim 5, wherein the first mesh of strands is a first knitting stitch configuration, and the second mesh of strands is a second knitting stitch configuration.

9. The spinal implant of claim 5, wherein the first mesh of strands comprises a first strand material, and the second mesh of strands comprises a second strand material different than the first strand material.

10. The spinal implant of claim 1, wherein the first chamber segment is a posterior chamber segment and the second chamber segment is an anterior chamber segment.

11. The spinal implant of claim 1, wherein the first chamber segment is a first lateral chamber segment and the second chamber segment is a second lateral chamber segment, wherein the second lateral chamber segment is disposed opposite the first lateral chamber segment.

12. A spinal implant configured to receive a fill material, comprising:
a wall that defines an interior chamber, wherein the wall has an interior side, an exterior side, and a wall thickness that extends between the interior side and the exterior side, wherein the wall has a porosity that permits fluid passage through the wall thickness and permits blood vessels and fibrous tissue to extend through the wall thickness; and
wherein the interior chamber has a first chamber segment and a second chamber segment, and in the first chamber segment the wall has a first mesh of strands configuration, and in the second chamber segment the wall has a second mesh of strands configuration.

13. The spinal implant of claim 12, wherein the first mesh of strands configuration is a first knitting stitch configuration, and the second mesh of strands configuration is a second knitting stitch configuration that is different than the first knitting stitch configuration.

14. The spinal implant of claim 13, wherein the first knitting stitch configuration is a first Jersey knitting stitch configuration, and the second knitting stitch configuration is a second Jersey knitting stitch configuration.

15. The spinal implant of claim 14, wherein the first Jersey knitting stitch configuration has a first set of stitch parameters and the second Jersey knitting stitch configuration has a second set of stitch parameters, and the first set of stitch parameters is different than the second set of stitch parameters.

16. The spinal implant of claim 12, wherein the first chamber segment has a first volumetric expansion ratio that is a function of the first mesh of strands configuration, and the second chamber segment has a second volumetric expansion ratio that is a function of the second mesh of strands configuration, wherein the first volumetric expansion ratio is different than the first volumetric expansion ratio.

17. The spinal implant of claim 12, wherein the first mesh of strands configuration comprises a first strand material, and the second mesh of strands configuration comprises a second strand material different than the first strand material.

18. The spinal implant of claim 12, wherein the first chamber segment is disposed to be a posterior chamber segment and the second chamber segment is disposed to be an anterior chamber segment.

19. The spinal implant of claim 12, wherein the first chamber segment is disposed to be a first lateral chamber segment and the second chamber segment is disposed to be a second lateral chamber segment, wherein the second lateral chamber segment is disposed opposite the first lateral chamber segment.

20. A spinal implant system, comprising:
a fill material;
an implant that comprises a wall that defines an interior chamber, wherein the wall has an interior side, an exterior side, and a wall thickness that extends between the interior side and the exterior side, wherein the wall has a porosity that permits fluid passage through the wall thickness and permits blood vessels and fibrous tissue to extend through the wall thickness; and
wherein the interior chamber has a first chamber segment and a second chamber segment, and in the first chamber segment the wall has a first wall configuration, and in the second chamber segment the wall has a second wall configuration; and
wherein the first chamber segment is configured to expand by a first volumetric expansion ratio when the fill material is packed into the first chamber segment in a manner that applies a first pressure to the wall within the first chamber segment, and the second chamber segment is configured to expand by a second volumetric expansion ratio when the fill material is packed into the second chamber segment in a manner that applies a second pressure to the wall within the second chamber segment, wherein the first pressure substantially equals the second pressure.
